(19) United States
(12) Patent Application Publication
(10) Pub. No.: US 2016/0184429 A1
(43) Pub. Date: Jun. 30, 2016

(54) SYSTEMS AND METHOD FOR THE TREATMENT OF BLADDER CANCER

(71) Applicants: LOMA LINDA UNIVERSITY MEDICAL CENTER, Loma Linda, CA (US); LOMA LINDA UNIVERSITY, Loma Linda, CA (US); NORTH CAROLINA STATE UNIVERSITY, Raleigh, NC (US)

(72) Inventors: Wolff M. Kirsch, Redlands, CA (US); Samuel M. Hudson, Raleigh, NC (US); Andrew Crofton, Loma Linda, CA (US)

(21) Appl. No.: 14/910,674

(22) PCT Filed: Aug. 7, 2014
(86) PCT No.: PCT/US2014/050188

§ 371 Date: Feb. 5, 2016

Related U.S. Application Data
(60) Provisional application No. 61/972,170, filed on Mar. 28, 2014.
(60) Provisional application No. 61/863,870, filed on Aug. 8, 2013.

(51) Int. Cl. A61K 41/00; A61K 47/36; A61K 9/51; A61K 38/20

(52) U.S. Cl. CPC A61K 41/0019; A61K 38/208; A61K 47/36; A61K 9/5161

(57) ABSTRACT

A chitosan material is treated in a nitrogen field by applying energy to ionize nitrogen in and around the chitosan, and the chitosan material is formulated into a hydrogel which can be utilized as a drug delivery vehicle for medicaments or therapeutic agents to treat certain conditions, such as cancers.

### CROSS-REFERENCE TO RELATED APPLICATIONS

This present application claims priority benefit under 35 U.S.C. §119(e) to U.S. Provisional Application No. 61/972,170, filed Mar. 28, 2014, entitled “SYSTEMS AND METHODS FOR THE TREATMENT OF BLADDER CANCER” and U.S. Provisional Application No. 61/863,870, filed Aug. 8, 2013, entitled “METHOD OF TREATMENT OF BLADDER CANCER” each of which are hereby incorporated by reference in their entirety.

### BACKGROUND OF THE INVENTION

1. Field of the Invention

Embodiments described herein relate to apparatuses, systems, and methods the treatment for bladder cancer in combination with depyrogenated chitosan.

2. Description of the Related Art

Prior art chitosan dressings suitable for use in internal applications have been difficult to produce, particularly chitosan materials that meet the necessary endotoxin levels allowable for implantable or internal medical devices. Further, when used internally, implantable treatments for bladder cancer have had biocompatablity problems, which have made such treatments undesirable.

### SUMMARY OF THE INVENTION

In a generally applicable first aspect (i.e. independently combinable with any of the aspects or embodiments identified herein), a method of making a drug delivery device, comprises irradiating endotoxin-containing chitosan under a nitrogen plasma in a presence of γ-irradiation, whereby an amount of endotoxins present in the chitosan is reduced; forming the irradiated chitosan into a hydrogel material; and combining a therapeutic agent with the hydrogel material, whereby a drug delivery device is obtained, wherein an amount of endotoxins present in the drug delivery device is less than 20 E.U. per device or less than 0.5 E.U. per gram. In an embodiment of the first aspect, which is generally applicable (i.e., independently combinable with any of the aspects or embodiments identified herein), irradiating is conducted at ambient temperature. In an embodiment of the first aspect, which is generally applicable (i.e., independently combinable with any of the aspects or embodiments identified herein), after the irradiating the chitosan is not substantially reduced in molecular weight. In an embodiment of the first aspect, which is generally applicable (i.e., independently combinable with any of the aspects or embodiments identified herein), the irradiating is conducted under γ-irradiation at 25 kGy for 15 hours. In an embodiment of the first aspect, which is generally applicable (i.e., independently combinable with any of the aspects or embodiments identified herein), the nitrogen-based plasma consists essentially of nitrogen plasma. In an embodiment of the first aspect, which is generally applicable (i.e., independently combinable with any of the aspects or embodiments identified herein), the nitrogen-based plasma consists of nitrogen plasma. In an embodiment of the first aspect, which is generally applicable (i.e., independently combinable with any of the aspects or embodiments identified herein), the therapeutic agent is interleukin-12. In an embodiment of the first aspect, which is generally applicable (i.e., independently combinable with any of the aspects or embodiments identified herein), the therapeutic agent is injected into the hydrogel material. In an embodiment of the first aspect, which is generally applicable (i.e., independently combinable with any of the aspects or embodiments identified herein), the therapeutic agent is combined with the hydrogel material prior to swelling of the gel.

In a generally applicable second aspect (i.e. independently combinable with any of the aspects or embodiments identified herein), a method of making a drug delivery device, comprises irradiating endotoxin-containing chitosan under a nitrogen plasma in a presence of γ-irradiation, whereby an amount of endotoxins present in the chitosan is reduced; forming the irradiated chitosan into a nanoparticle; and encapsulating a therapeutic agent within the nanoparticle, whereby a drug delivery device is obtained, wherein an amount of endotoxins present in the device is less than 20 E.U. per device or less than 0.5 E.U. per gram. In an embodiment of the second aspect, which is generally applicable (i.e., independently combinable with any of the aspects or embodiments identified herein), the therapeutic agent is interleukin-12. In an embodiment of the second aspect, which is generally applicable (i.e., independently combinable with any of the aspects or embodiments identified herein), irradiating is conducted at ambient temperature. In an embodiment of the second aspect, which is generally applicable (i.e., independently combinable with any of the aspects or embodiments identified herein), after the irradiating the chitosan is not substantially reduced in molecular weight. In an embodiment of the second aspect, which is generally applicable (i.e., independently combinable with any of the aspects or embodiments identified herein), the irradiating is conducted under γ-irradiation at 25 kGy for 15 hours. In an embodiment of the second aspect, which is generally applicable (i.e., independently combinable with any of the aspects or embodiments identified herein), the nitrogen-based plasma consists essentially of nitrogen plasma. In an embodiment of the second aspect, which is generally applicable (i.e., independently combinable with any of the aspects or embodiments identified herein), the nitrogen-based plasma consists of nitrogen plasma.

In a generally applicable third aspect (i.e. independently combinable with any of the aspects or embodiments identified herein), a pharmaceutical composition, comprising: a hydrogel of chitosan; and a therapeutic agent, wherein the hydrogel is configured to deliver the therapeutic agent to a target tissue, and wherein an amount of endotoxins present in the pharmaceutical composition is less than 0.5 E.U. per gram. In an embodiment of the third aspect, which is generally applicable (i.e., independently combinable with any of the aspects or embodiments identified herein), the therapeutic agent is interleukin-12. In an embodiment of the third aspect, which is generally applicable (i.e., independently combinable with any of the aspects or embodiments identified herein), the chitosan of the hydrogel is derived from an endotoxin-containing chitosan that is irradiated under a nitrogen plasma in a presence of γ-irradiation so as to reduce the amount of endotoxins present in the device to less than 20 E.U. per device or 0.5 E.U. per gram. In an embodiment of the third aspect, which is generally applicable (i.e., independently combinable with any of the aspects or embodiments identified herein), the nitrogen plasma consists essentially of nitrogen plasma. In an embodiment of the third aspect, which is generally applicable (i.e., independently combinable with any of the aspects or embodiments identified herein), a molecular weight of the chitosan is not substantially reduced upon irradiation. In an embodiment of the third aspect, which is generally applicable (i.e., independently combinable with any of the aspects or embodiments identified herein), an average molecular weight of the chitosan is not reduced more than 5% upon irradiation.

In a generally applicable fourth aspect (i.e. independently combinable with any of the aspects or embodiments identified herein), a drug delivery device comprising the pharmaceutical composition comprising: a hydrogel of chitosan; and a therapeutic agent, wherein the hydrogel is configured to deliver the therapeutic agent to a target tissue, and wherein an amount of endotoxins present in the pharmaceutical composition is less than 0.5 E.U. per gram, wherein an amount of endotoxins present in the drug delivery device is less than 20 E.U. per device.

Any of the features of an embodiment of the first through fourth aspects is applicable to all aspects and embodiments identified herein. Moreover, any of the features of an embodiment of the first through third aspects is independently combinable, partly or wholly with other embodiments described herein in any way, e.g., one, two, or three or more embodiments may be combinable in whole or in part. Further, any of the features of an embodiment of the first through third aspects may be made optional to other aspects or embodiments. Any aspect or embodiment of a method can be performed by a system or apparatus of another aspect or embodiment, and any aspect or embodiment of a system can be configured to perform a method of another aspect or embodiment.

### BRIEF DESCRIPTION OF THE DRAWINGS

FIG. 1 schematically depicts a process for obtaining chitosan from crustacean shell waste in accordance with one embodiment.

FIG. 2 schematically depicts an embodiment of an apparatus for preparing chitosan fibers.

FIG. 3 provides a schematic of an assembly line for production of chitosan fleece in accordance with one embodiment.

FIG. 4A is a scanning electron microscope image of a microfibrillar chitosan prepared in accordance with one embodiment.

FIG. 4B is an edge enhanced image of FIG. 4A.

FIG. 5 is a schematic depiction of one embodiment of a plasma treatment assembly.

FIGS. 6A-D illustrate an Instron setup for testing of bioadhesivity of chitosan before and after electron beam sterilization.

FIGS. 7A-B show the bioadhesion of chitosan to chicken gizzard measured in force.

### DETAILED DESCRIPTION OF THE PREFERRED EMBODIMENT

Chitosan is obtained from chitin, a widely available biopolymer obtained principally from shrimp and crab shell waste. Chitosan is the main derivative of chitin, and is the collective term applied to deacetylated chitins in various stages of deacetylation and depolymerization. The chemical structure of chitin and chitosan is similar to that of cellulose. The difference is that instead of the hydroxyl group as is bonded at C-2 in each D-glucose unit of cellulose, there is an acetylated amino group (—NHCOCH₃) at C-2 in each D-glucose unit in chitin and an amino group at C-2 in each D-glucose unit of chitosan.

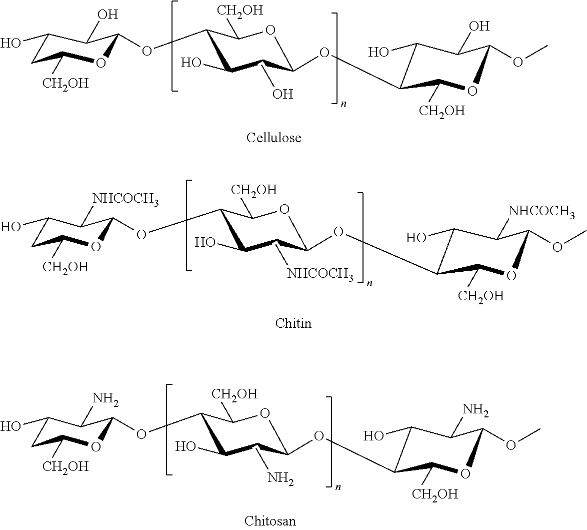

Chitin and chitosan are both nontoxic, but chitosan is used more widely in medical and pharmaceutical applications than chitin because of its good solubility in acid solution. Chitosan has good biocompatibility and is biodegradable by chitosanase, papain, cellulase, and acid protease. Chitosan can exhibit anti-inflammatory and analgesic effects, and promotes hemostasis and wound healing. Chitosan has also been shown to be an effective hemostatic agent. Chitosan hemostasis is believed to be mediated by positively charged amine groups binding to negatively charged red cell and platelet surfaces forming a mucoadhesive coagulum without activation of classical coagulation pathways.

In a preferred embodiment, a chitosan device made from microfibrillar high molecular weight chitosan can be constructed in the form of sponge, puff or non-woven fabric. The microfibrillar high molecular weight chitosan is discussed in Applicants' copending U.S. application Ser. No. 10/868,201, filed Mar. 12, 2013, and directed to a “DEPLOYABLE MULTIFUNCTIONAL HEMOSTATIC AGENT”, published as U.S. Publ. No. 2005/0123588 A1, and copending U.S. application Ser. No. 11/061,243, filed Feb. 18, 2005, and directed to a “HEMOSTATIC AGENT FOR TOPICAL AND INTERNAL USE”, published as U.S. Publ. No. 2005/0240137 A1. The entirety of both of these copending applications, and particularly the disclosure directed to making and using chitosan-based hemostatic devices, is hereby incorporated by reference.

Additionally, chitosan can be an effective drug delivery vehicle due to the anti-inflammatory and analgesic effects. Chitosan can also be an effective drug delivery vehicle due to its ability to adhere to tissues, loosen gap junctions, and incorporate therapeutic compounds under mild conditions. The use of chitosan nanoparticles as a drug delivery device to deliver inhibitors (alphaGal lectin, anti-CS Mab, C1-Inhibitor, factor H, human CD59 cDNA) to the brain for treatment of cerebral amyloid angiopathy is discussed in Applicants' copending International Patent Application No. PCT/US2013/030582, filed Mar. 12, 2013, and directed to a “SUBSTANCES AND METHODS FOR THE TREATMENT OF CEREBRAL AMYLOID ANGIOPATHY RELATED CONDITIONS OR DISEASES”, now published as WO 2013/138368. The entirety of this application is hereby incorporated by reference.

As discussed above, chitosan is formed from chitin, which is present in crustacean shells as a composite with proteins and calcium salts. Chitin is produced by removing calcium carbonate and protein from these shells, and chitosan is produced by deacetylation of chitin in a strong alkali solution.

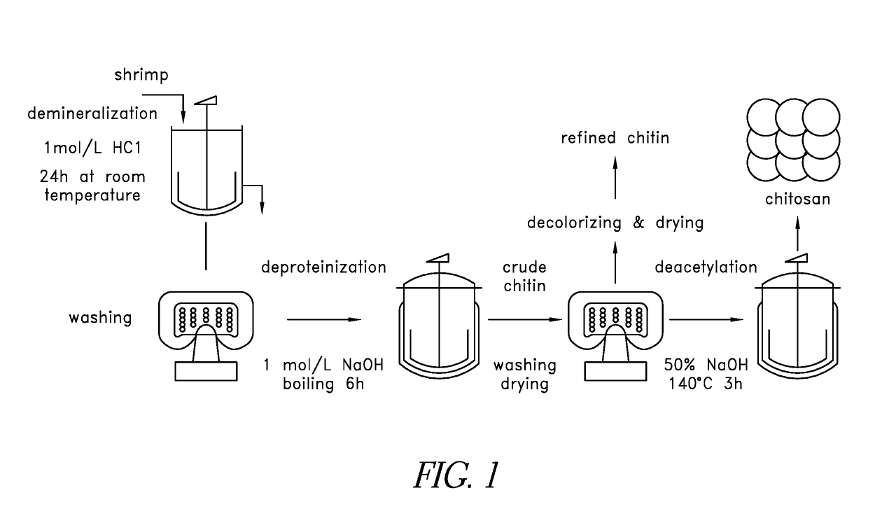

One method for obtaining chitosan from crab, shrimp or other crustacean shells, including Dungeness crab shells, is schematically depicted in FIG. 1 and described as follows. Calcium carbonate is removed by immersing the shell in dilute hydrochloric acid at room temperature for 24 hours (demineralization). Proteins are then extracted from the decalcified shells by boiling them with dilute aqueous sodium hydroxide for six hours (deproteinization). The demineralization and deproteinization steps are preferably repeated at least two times to remove substantially all of the inorganic materials and proteins from the crustacean shells. The crude chitin thus obtained is washed then dried. The chitin is heated at 140° C. in a strong alkali solution (50 wt. %) for 3 hours. Highly deacetylated chitosan exhibiting no significant degradation of molecular chain is then obtained by intermittently washing the intermediate product in water two or more times during the alkali treatment.

### Chitosan Fibers and Endotoxin Removal

Chitosan fibers can be prepared by a wet spinning method, although any suitable method could be used. In one embodiment, chitosan is first dissolved in a suitable solvent to yield a primary spinning solution. Solvents can include acidic solutions, for example, solutions containing trichloroacetic acetic acid, acetic acid, lactic acid, or the like; however any suitable solvent can be employed. The primary spinning solution is filtered and deaerated, after which it is sprayed under pressure into a solidifying bath through the pores of a spinning jet. Solid chitosan fibers are recovered from the solidified bath. The fibers can be subjected to further processing steps, including but not limited to drawing, washing, drying, post treatment, functionalization, and the like.

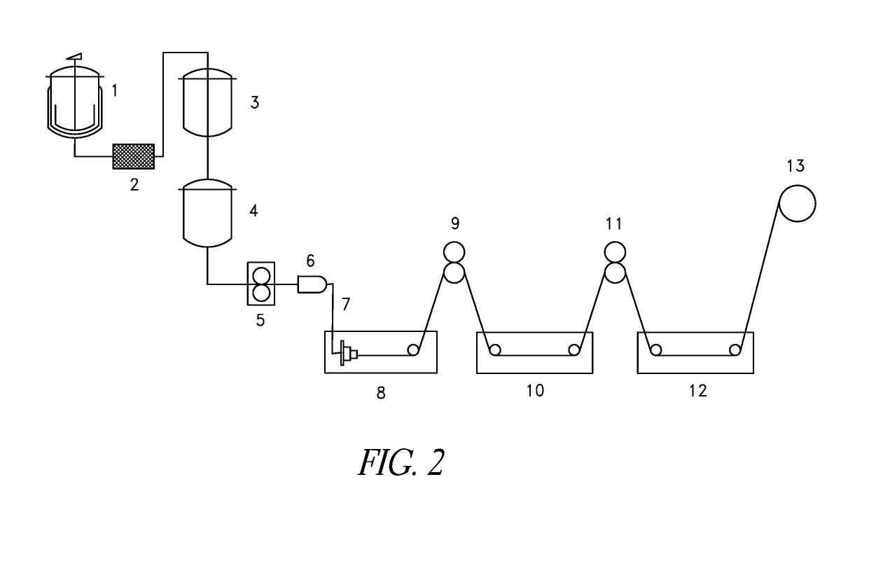

FIG. 2 illustrates an apparatus for preparing chitosan fibers in accordance with one embodiment. The illustrated apparatus includes a dissolving kettle 1, a filter 2, a middle tank 3, a storage tank 4, a dosage pump 5, a filter 6, a spinning jet 7, a solidifying bath 8, a pickup roll 9, a draw bath 10, a draw roll 11, a washing bath 12, and a coiling roll 13.

In one embodiment, the primary chitosan spinning solution is prepared by dissolving 3 parts chitosan powder in a mixed solvent at 5° C. containing 50 parts trichloroacetic acid (TDA) to 50 parts methylene dichloride. The resulting primary spinning solution is filtered and then deaerated under vacuum. A first solidifying bath comprising acetone at 14° C. is employed. The aperture of the spinning jet is 0.08 mm, the hole count is forty-eight, and the spinning velocity is 10 m/min. The spinning solution is maintained at 20° C. by heating with recycled hot water. The chitosan fibers from the acetone bath are recovered and conveyed via a conveyor belt to a second solidifying bath comprising methanol at 15° C. The fibers are maintained in the second solidifying bath for ten minutes. The fibers are recovered and then coiled at a velocity of 9 m/min. The coiled fibers are neutralized in a 0.3 g/l KOH solution for one hour, and are then washed with deionized water. The resulting chitosan fiber is then dried.

In one embodiment, glacial, or anhydrous, acetic acid is employed as an agent to adhere the chitosan fibers to each other in embodiments where chitosan fibers, either alone or with an added medicament, therapeutic agent or other agent, are used in forming a hemostatic agent. In addition to providing good adherence between the chitosan fibers, fibers treated with glacial acetic acid also exhibit exceptional ability to adhere to wounds, including arterial or femoral wounds.

Depending upon the application, the concentration of acetic acid in solution can be adjusted to provide the desired degree of adhesion. For example, it can be desirable to employ a reduced concentration of acetic acid if the chitosan fibers are to be employed in treating a seeping wound or other wound where strong adhesion is not desired, or in applications where the hemostatic agent is to be removed from the wound. In such embodiments, an acetic concentration of from about 1 vol. % or less to about 20 vol. % is generally employed, and more preferably a concentration of from about 2, 3, 4, 5, 6, 7, 8, 9, or 10 vol. % to about 11, 12, 13, 14, 15, 16, 17, 18, or 19 vol. % is employed. Where strong adhesion between fibers, or strong adhesion to the wound is desired, a concentration greater than or equal to about 20 vol. % is preferred, more preferably from about 50, 55, 60, 65, or 70 vol. % to about 75, 80, 85, 90, 95, or 100 vol. %, and most preferably from about 95, 96, 97, 98, or 99 vol. % to about 100 vol. %.

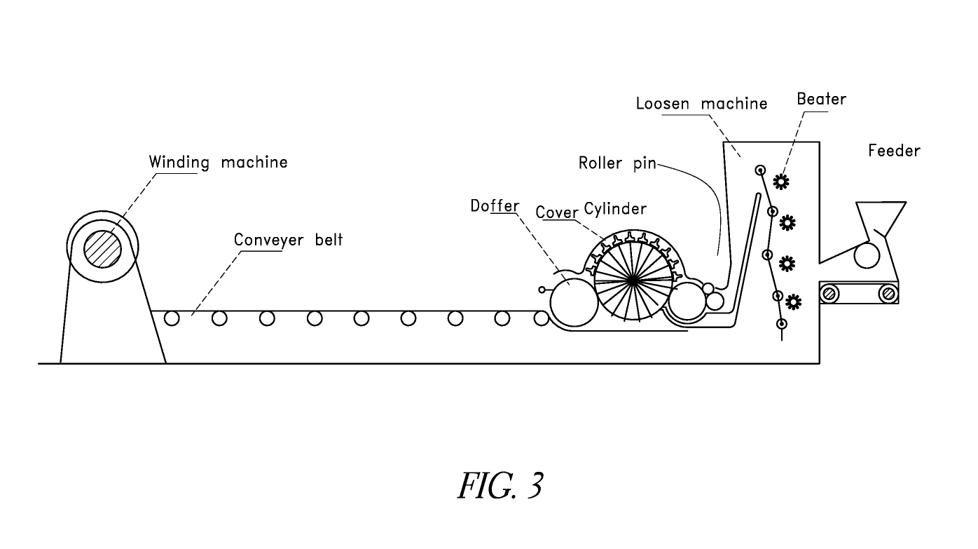

Chitosan textile can be prepared from chitosan fibers using equipment commonly employed in the textile industry for fiber production. With reference next to FIG. 3, an assembly line for production of chitosan fleece can employ a feeder, a loosen machine, a carding machine, a conveyor belt, and lastly a winding machine, as depicted below. In the feeder, chitosan short fiber is fed through a feeder and into a loosen machine, wherein chitosan short fiber is loosened by several beaters. In the carding machine, chitosan fibers are ripped and turned into chitosan fleece by high speed spinning of a cylinder and roller pin, then the fleece is peeled off as a separated thin layer of net by a duffer.

The production of fibers and associated processing discussed above can be effective when using chitosan of relatively high molecular weight. Such high molecular weight chitosan is amenable to formation into fibrous forms such as fleece that can be formed into a strong and durable textile that is flexible and malleable but retains continuity so that it can be moved as a unit and doesn't break apart when manipulated during use. In some embodiments, chitosan fibers can be formed into a yarn, which in turn can be woven. In other embodiments, successive layers of chitosan fiber pieces can be flattened and sprayed with an acidic solution (preferably a solution with a pH of about 3.0-4.5) such as the glacial acetic acid discussed above so as to form a non-woven textile.

In some embodiments, a fibrous hemostatic device is constructed of high molecular weight chitosan (<600 kDA). The high molecular weight chitosan lends itself to construction of a dry, fibrous hemostatic material that can be constructed as a textile in a puff, fleece, fabric or sheet form. In some embodiments, a fibrous hemostatic device can be constructed of chitosan with standard molecular weight. Such chitosan devices, similar to high molecular weight chitosan, can be amenable to formation into fibrous forms of strong durable textiles that are flexible and malleable but retain continuity to be moved as a unit and not break apart when manipulated during use or for treatments as discussed herein.

In some embodiments, microfibrillar chitosan, nanoparticulate chitosan, chitosan materials in the form of a hydrogel, and other chitosan materials known in the art can be amenable to all of these applications and configurations, and embodiments envisioned in which devices made from such chitosan are formed and shaped accordingly. Normally, however, chitosan is laden with pyrogens, particularly endotoxins, which can limit its applicability in the biological and medical arenas, as minute amounts of endotoxins may induce septic responses when contacted with mammalian tissue. As such, in accordance with some embodiments, a microfibrillar high molecular weight chitosan hemostat is used externally so as to minimize the likelihood of a septic response. In other embodiments, such chitosan hemostats can be used during surgeries, but only for temporary purposes, and are not implanted or left within a patient.

Endotoxins are essentially the skeletal or cellular remains and by-product secretions of dead bacteria, which are ubiquitous and found in the air, on surfaces and in food and water. More precisely, endotoxins are complex amphiphilic lipopolysaccharides (LPS) having both polysaccharide and lipophilic components. They are composed of pieces of the lipopolysaccharide wall component of Gram-negative bacteria. An example of LPS is shown below.

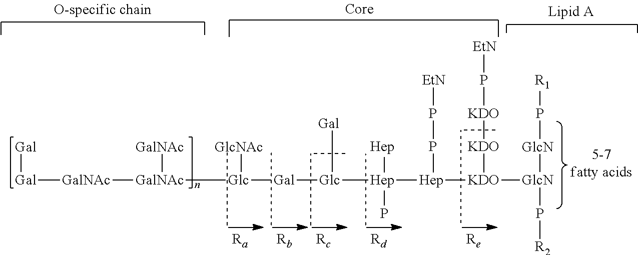

The terms endotoxin and pyrogen are often used interchangeably. Endotoxins are one of many pyrogens, which are substances that elicit a fever response in the bloodstream of a mammalian body. Vascular or lymphatic exposure to endotoxins can lead to severe sepsis, septic shock, and potential death. Thus, endotoxins are of particular concern to those manufacturing medical devices as they are one of the most potent pyrogens that can contaminate a product.

As such, pharmaceuticals, medical devices and products that contact human tissue, blood, bone or that can be absorbed by the body or implanted within the body must meet stringent levels of endotoxin control. The US Pharmacopeia set forth specifications for endotoxin units (EU) for medical devices and pharmaceuticals. The current standard (USP27) specifies <20 EU per device (e.g. <0.5 EU/mL in water) and/or <0.5 EU per gram. Various embodiments of chitosan-based hemostats and/or other chitosan-based materials anticipated for internal use have sufficiently reduced levels of endotoxins to comply with such standards. In the context of the embodiments, a “device” can include a unit dosage form of a liquid or solid pharmaceutical composition, e.g., a capsule, a tablet, a bolus, or the like, or multiple dosages intended to be administered sequentially or simultaneously, wherein an aggregate of the multiple dosages is a total dose to be administered to a patient in one treatment. Additionally, for example, a unit dosage of a pharmaceutical composition could require a maximum endotoxin load of <0.5 EU per gram for the chitosan-based pharmaceutical.

In some embodiments, multiple devices and/or multiple doses of the pharmaceutical can be administered at one time or within a certain timeframe. The cumulative endotoxin level for all devices cannot exceed the USP27 standard of <20 EU per device (e.g. <0.5 EU/mL in water) and/or <0.5 EU per gram. Therefore, in some embodiments, the endotoxin amount can influence the number of devices that can be implanted at one time. For example, 2 devices that contain 10 EU/device could be implanted in one surgery (20 EU total). For pharmaceuticals, since they are given at varying time intervals, the endotoxin quantity can influence the amount of drug that can be given in a certain window of time. Additionally, in some embodiments, the pharmaceutical composition with chitosan can be a 2% solution which will reduce the amount of endotoxins in the chitosan solution enabling a large dosage to be given in the event that endotoxin contamination levels are a concern.

Further, in some embodiments, the allowable endotoxin limits for internal uses of a device or pharmaceutical over time can be based on characteristics of the patient, for example the patient's height and/or weight. The endotoxin effect within the body is related to an immune system response to the presence of endotoxins. Therefore, once the endotoxin load is processed and/or expelled from the body and the body has substantially cleared the endotoxins from the previous implantation or administration additional devices and/or doses containing endotoxins can be introduced into the body. The endotoxins present in the patient due to the medical device or pharmaceutical can be expelled from the body through normal body processes. In some embodiments, the endotoxins can be processed and expelled within between about 12 hours to about 3 weeks. For example, the endotoxins can be processed and expelled within about 12 hours, about 1 day, about 3 days, about 1 week, about 2 weeks, or about 3 weeks.

Endotoxins are notoriously difficult to remove from materials. They are extremely resilient; they are strong, tough and elastic, remain viable after steam sterilization and normal desiccation, and can pass through filters. Research shows that temperatures in excess of 200° C. for up to an hour can be required to remove endotoxin contamination.

As endotoxins are ubiquitous in biological materials, much effort and research has been dedicated to removal and/or inactivation of endotoxins in order to make biological materials useful for medical purposes. Some of the treatment methods that have been researched and employed include heat, acid base hydrolysis, oxidation, ionizing radiation such as gamma-irradiation, and ultra-filtration. These methods have varying ranges of effectiveness, expense, and suitability for particular products.

It has proven difficult, however, to develop an endotoxin removal or inactivation process (depyrogenation) that is suitable for chitosan, particularly high molecular weight chitosan, as known processes such as contacting the chitosan with a strong base or γ-irradiating aqueous chitosan solutions tends to depolymerize the chitosan, resultingly decreasing the average molecular weight.

As discussed above, various embodiments of a chitosan-based hemostatic textile and other chitosan-based materials described herein or known in the art can employ chitosan having very high molecular weight. Obtaining such chitosan involves important choices and procedures. A particularly preferred source of chitin for use in preparing embodiments of chitosan textiles is crab shell, including Dungeness crab shells. Chitin prepared from crab shell, particularly arctic crab shell, generally exhibits a molecular weight that is much higher than the molecular weight of chitin made from shrimp shell. Crab shell chitin also generally exhibits a higher degree of deacetylation than shrimp shell chitin. Crab shell chitin typically exhibits an average molecular weight of from about 600-1,300 kDa. Such high molecular weight chitosan can more readily be processed to form sturdy fibers.

Preferred chitin material for use in preparing chitosan fiber in accordance with some embodiments has a molecular weight of greater than about 600, 650, 700, 750, 800, 850, 900, 950, 1000, 1100, 1200, 1300, 1400, or 1500 kDa or more; more preferably a molecular weight in a range from about 600-800 kDa; and most preferably about 700 kDa. Preferably, resulting chitosan fibers have similar molecular weights. Preferably, the chitosan preferably has a degree of deacetylation in a range between about 75-90%, more preferably in a range between about 80-88%, and most preferably in a range between about 80-85%.

In accordance with an embodiment, arctic crab shells such as Alaska snow crab shells are used as the raw material for microfibrillar chitosan. These shells preferably are washed, crushed, dried, then soaked for 12 hours in 3-5% HCl for 1-2 hours to demineralize and deproteinize the material. The slurry is transferred into a 5% NaOH reactor at 90° C. for another protein removal. Deproteinized crushed shells are washed twice with water until neutral, dried and decolorized again by exposure to ultraviolet light. Another decalcification and deproteinization follows for 12 hours in 3% HCl, followed by 3-5% NaOH 90° C. for another 1-2 hours. The deproteinized, demineralized material is washed by water to neutrality, dried and UV decolorized. At this stage the shell material has been processed to the form of chitin, and has a residual protein level ≦0.1%, which is significantly lower than commodity grade chitosan.

To process the chitin to high molecular weight chitosan in accordance with one embodiment, the material is subjected to controlled deacetylation in a 48% NaOH solution at 90° C. for 4 hours. Preferably, the degree of deacetylation (DA) is monitored by titration method to 80-88%, and more preferably about 85% as mentioned above, in order to produce high molecular weight (M.W.) chitosan (M.W. >600 kDa). Also, as noted above, crab shell chitin is unique in providing high molecular weight chitosan. Applicants have determined that high molecular weight chitosan provides a significant advantage for both endotoxin/pyrogen reduction and microfiber production in order to facilitate construction of a chitosan-based textile.

To process high molecular weight chitosan (in various embodiments a high molecular weight is considered to be ≧600 kDa) in accordance with a preferred embodiment, the chitosan is dissolved in 1% trichloroacetic acid, filtered, deaerated and forced under pressure into a solidifying bath through the pores of a spinning jet (the spinneret pack). Chitosan fibers recovered from the solidified bath are washed, dried, and collected as fibers in a solidifying acetone bath (14° C.). The aperture of the spinning jet preferably is 0.8 mm (800 microns), hole count 48, and spinning velocity 10 m/min. 20° C. Chitosan fibers from the acetone bath are moved by conveyor belt to a second solidifying bath (methanol at 15° C.). Fibers are maintained in the second solidifying bath for 10 minutes, recovered, and coiled at a velocity of 9 m/min. Coiled fibers are neutralized in a 0.3 g/L KOH solution for 1 hour before washing with deionized water, then dried, packaged and quarantined until cleared by analysis.

Chitosan processed as just discussed has been analyzed to yield the specifications as depicted in the below table, which specifications conform to the following guidelines: “ASTM F2103-01 Standard Guide for Characterization and Testing of Chitosan Salts as Starting Material Intended for Use in Biomedical and Tissue Engineered Medical Product Applications.”

| Item | Specification |
|---|---|
| Bioburden, aerobic count | A total aerobic count less than |
| | 500 cfu/gram. Total |
| | aerobic, fungi, spores and |
| | obligate anaerobes under |
| | 1000 cfu/gram |
| Degree of Deacetylation | 85% |
| Average Molecular Weight | 700,000 Daltons |
| pH of H₂O—C₂H₅OH Aq. | 5 ± 0.5 |
| Heavy Metals: Pb, Cr, Hg, Cd, As | ≦20 ppm |
| | <20 ppm total |
| Weight Loss on Drying | <15% |
| Color | White to slight yellow |
| Extractable Material | <0.1% protein |
| Solubility in Acid | <0.5% non-soluble in 1% acetic acid |
| Identity | FTIR |
| Bulk Packaging for Shipping | Sealed in metalized foil bags |
| | under nitrogen |
| Residual Protein | <1% |

Included Specifications after Microfiber,
Non-woven Fabric Production

| | |
|---|---|
| Fiber Denier | Range 9.1-26.9 micron O.D. |
| In vitro adhesion | Adhesive strength (kPa ~70-80) |
| Chitosan structure | No change in IR spectrum after UV |

Preferably, handling and storage of the manufactured chitosan product is conducted in an endotoxin-reduced, UV irradiated environment. All bags, containers, and storage materials preferably are pyrogen free and the product is stored and transferred in a nitrogen atmosphere.

Applicants have found that high molecular weight chitosan as discussed above has less of an affinity for endotoxins than low molecular weight chitosan. Thus, although a need to inactivate endotoxins likely still exists, the high molecular weight chitosan is more amenable to successful inactivation treatment.

In one embodiment, end-product high molecular weight fibrous chitosan fleece was packaged under nitrogen. In some such embodiments, the fleece is packaged in a container made of olefin fibers such as Tyvek™. In some embodiments the packaging comprises a plastic material with or without a thin metalized layer. It is anticipated that other types of packaging may be employed. Preferably, however, the packages are sealed, keeping the fleece in an environment of nitrogen gas, and preventing entry by oxygen.

In another embodiment, packages having high molecular weight fibrous chitosan fleece prepared as discussed above and sealed in a nitrogen field such as just discussed can be irradiated with γ-irradiation (CO⁶⁰ source) at 25 kGy over 15 hours. It is anticipated and understood that other doses and intensities of γ-irradiation can be employed. However, Applicants tested chitosan fleece so prepared by implantation into rabbits to monitor the toxic response and thus evaluate the effectiveness of γ-irradiation in inactivating endotoxin contamination in high molecular weight chitosan. Applicants noted the septic response to the γ-irradiated chitosan was markedly less than that of the non-irradiated chitosan as implanted into the same rabbit. More particularly, non-irradiated chitosan exhibited substantial pus formation and localized necrosis and inflammation, while the γ-irradiated sample showed little to none of these effects.

Chitosan is graded by “purity,” ranging from impure “food” or “commodity grade” to highly purified “medical grade.” To qualify as “medical grade” chitosan endotoxin/pyrogen levels have to be reduced as designated by the FDA and U.S. Pharmacopeia. The endotoxin standards (USP27) for FDA approval of implantable medical devices (chitosan hemostats) are <20 EU (endotoxin units) per device or <0.5 EU/ml in water. Since endotoxin molecular weights vary (10,000 to 10⁶ Da), quantitation is measured as EU, where one EU is equivalent to 100 pg of E. coli lipopolysaccharide (LPS). These levels are typically measured by the Limulus Amoebocyte Lysate (LAL) test.

Applicants sent six samples of high molecular weight chitosan samples prepared as discussed above and γ-irradiated under nitrogen for LAL testing, along with six samples that had not been irradiated. The samples were prepared as summarized below:

### Sample

### Preparation:

Samples were cut and immersed:

| | | | |
|---|---|---|---|
| Extraction Method: | X | Immersion | Fluid Pathway |

| | |
|---|---|
| No. of Samples: | 6 |
| Total Extraction Volume: | 60.0 mL |
| Static Soak Time: | 60 minutes |
| Extraction Temperature: | 20-25° |

The samples were then tested to detect the concentrations of EUs per device. Since certain properties of endotoxins often interfere with the results of undiluted samples, endotoxins were measured at stepped levels of dilution, with anticipated results becoming more reliable with successive dilutions. The test results follow below:

| | | |
|---|---|---|
| ENDOTOXIN UNITS (EU) PER | Undiluted | 20.70 EU/Device |
| DEVICE: | 2 fold | 18.40 EU/Device |
| | 10 fold | 9.77 EU/Device |
| | 20 fold | 8.60 EU/Device |

As indicated in the test results, the reliable 10 fold and 20 fold diluted test samples yield levels of EU/Device that are well within the acceptable limits for medical grade, implantable chitosan.

In contrast, the six samples that were NOT irradiated were prepared in a similar manner, yet yielded the following test results:

| | | |
|---|---|---|
| ENDOTOXIN UNITS (EU) PER | Undiluted | >50.00 EU/Device |
| DEVICE: | 2 fold | 70.00 EU/Device |
| | 10 fold | 68.80 EU/Device |
| | 20 fold | 73.00 EU/Device |

The 10 fold and 20 fold diluted sample tests show levels of endotoxin EU that are well beyond the acceptable maximum levels of endotoxin EU for medical grade chitosan. As the only difference in the samples was γ-irradiation in a sealed package in a nitrogen environment, Applicants have concluded that γ-irradiation of high molecular weight chitosan under these conditions effectively inactivates endotoxins. Additionally, testing of the γ-irradiated chitosan against non-irradiated chitosan for hemostatic efficacy resulted in no detectable difference.

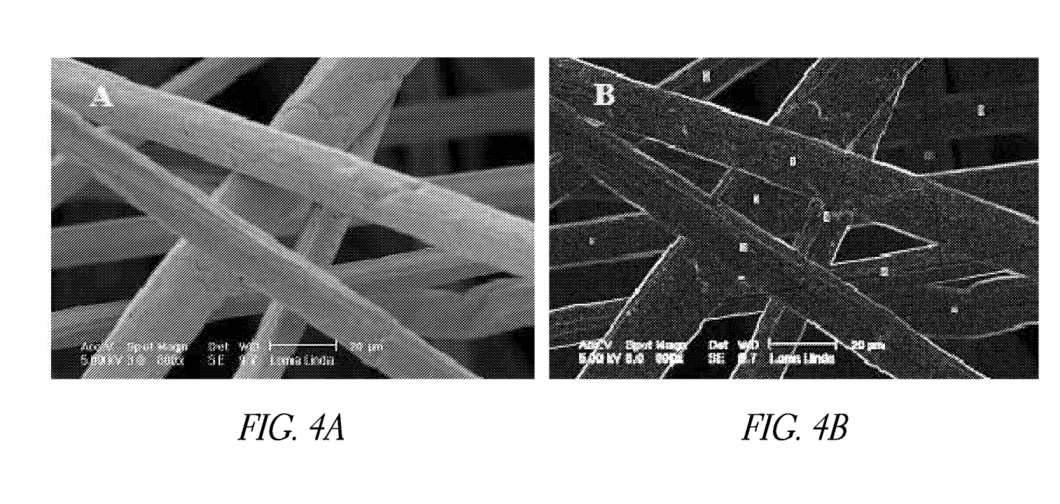

The samples were further investigated to determine whether the γ-irradiation had caused depolymerization and/or otherwise damaged the chitosan fibers. The images in FIGS. 4A and 4B depict Scanning Electron Microscopy (SEM) surface areas of microfibrillar chitosan processed as described above and irradiated as discussed above. FIG. 4A is a SEM of microfibrillar chitosan, mean diameter of fibers 16.7±3.6 μm (range 10-26 μm). FIG. 4B is an edge enhanced image of FIG. 4A, created and analyzed using ImageJ software (ImageJ, NIH). Eleven fibers in the 150×100 μm field of view (FOV) were modeled as cylinders using fiber length and width estimates from the image. The surface area to volume ratio (S/Vₚ) of microfibrillar chitosan using the FOV dimensions and assuming a depth of six times the average fiber diameter (16.7 μm), is 4.7 nm⁻¹. Therefore, a dressing thickness and blood penetration depth of 5 mm, a 1×1×5 mm volume of microfibrillar chitosan presents an estimated surface area of 23.5 μm² to blood products.

In summary, the irradiated chitosan fibers were structurally intact, and maintained a high surface area that was available for interaction with blood. Applicants have concluded that the irradiation under the listed conditions caused little to no depolymerization and/or reduction in molecular weight of the chitosan fibers.

The high molecular weight chitosan fibers prepared as discussed above have a relatively high nitrogen content. Applicants have determined that treating such fibers in conditions conducive to ionization of nitrogen is especially beneficial in inactivating endotoxin without substantially damaging the chitosan fiber structure. More particularly, in some embodiments, preferably chitosan is subjected to a treatment that increases the quantity of amino groups in and around fibrous chitosan, and even more preferably a treatment that creates nitrogen-based free radicals, so as to inactivate endotoxin and simultaneously increase one or more of wettability, hydrophilicity, and mucoadhesion.

In another embodiment, a high molecular weight chitosan is treated with an ionized nitrogen gas, more specifically a nitrogen-based plasma, preferably under ambient temperature, so as to effectively inactivate endotoxins on high molecular weight chitosan without negatively affecting the efficacy or molecular weight of the chitosan.

In one embodiment, plasma treatment can be carried out using, for example, an e⁻Rio™ atmospheric pressure plasma system APPR-300-13 available from APJeT Inc. The machine uses RF electric fields, 1300 W @ 27 MHz RF/1 mm gap, to produce a unique, non-thermal, glow-discharge plasma that operates at atmospheric pressure with a cooling requirement of 1 gpm @ 20 psi max.

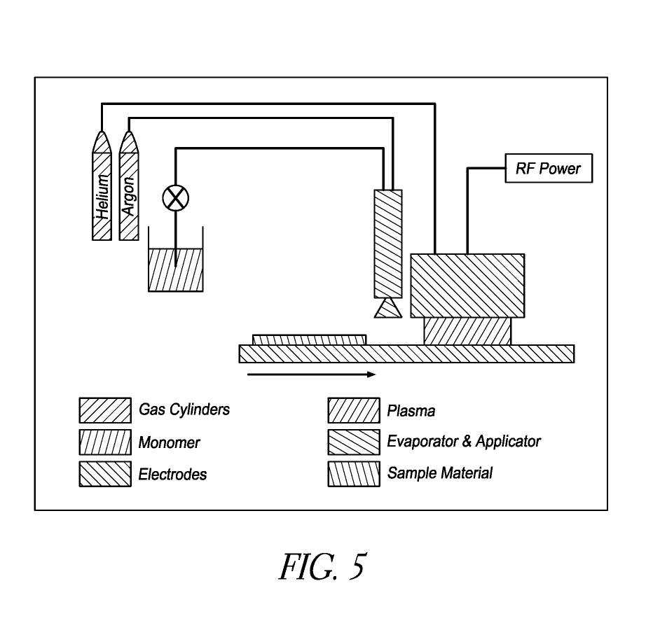

With reference next to the exemplary schematic in FIG. 5, in some embodiments the plasma assembly will include an evaporator and applicator. The evaporator is a heated assembly that vaporizes a monomer that is to be applied to fibrous chitosan samples. Heat is regulated by a logic controller that is connected to a thermo-coupler attached to the evaporator. The applicator acts as a heated nozzle to apply vaporized monomer to the fibrous chitosan sample. The heat maintains the vapor property of the monomer. Heat preferably is regulated by a logic controller that is connected to a thermo-coupler attached to the applicator.

It is to be understood that multiple methods and assemblies for plasma treatment of high molecular weight chitosan can be employed. For example, fibrous chitosan and/or other forms of chitosan disclosed herein can be treated under a nitrogen plasma and then packaged under nitrogen gas. In some embodiments, relatively large quantities of fibrous chitosan are treated under nitrogen plasma and are then divided into individual doses and packaged separately. In still other embodiments, chitosan can be partially packaged, such as enclosed within a package having an unsealed opening, plasma-treated in the partially packaged condition, and the package may be fully sealed in the plasma treatment zone or a nearby nitrogen field. In some embodiments, chitosan materials can be packaged in Tyvek® pouches under nitrogen gas, sealed, and subsequently treated with plasma.

In further various embodiments, high molecular weight chitosan can be packaged prior to plasma treatments. Preferably the chitosan textile or other chitosan material can be sealed in a nitrogen field, and can be prepared substantially as discussed above. In some such embodiments, the RF power activates the nitrogen within the packaging, which is believed to create nitrogen-based free radicals that contribute to deactivation of the endotoxin. Of course, it is to be understood that various types and configurations of assemblies and apparatus may be used for the plasma treatment.

Embodiments discussed above have described treating fibrous high molecular weight chitosan and/or other chitosan materials described herein or known in the art in a nitrogen field involving plasma, γ-irradiation, or the like. In other embodiments, other methods and apparatus that will increase the concentration of amino groups on and around the chitosan can be employed. Preferably such methods additionally provide nitrogen-based free radicals. Such methods may involve other types of irradiation, as well as variations in power, duration, and the like as compared to the examples specifically discussed herein.

In accordance with yet further embodiments, high molecular weight chitosan is treated using both plasma and a nitrogen field and γ-irradiation. In some embodiments the chitosan is first treated γ-irradiation and then treated under the plasma. In other embodiments the order is reversed.

Applicants treated samples of fibrous high molecular weight chitosan having a molecular weight about 700 kDa and a degree of acetylation of about 85%, which samples had been sealed in packages and in a nitrogen field, by first γ-irradiating the packaged samples at a level of 25 Gy, and then plasma treating the still-packaged samples. The treated samples were then subjected to LAL testing. A sample so treated under plasma for about 5 minutes was tested to have 9.6 EU/device, and 52.8 EU/g based on a 20-fold dilution. A sample so treated under plasma for about 10 minutes was tested to have 2.3 EU/device, and 12.7 EU/g based on a 20-fold dilution.

In some embodiments described above, fibrous chitosan is treated with an acetic acid solution so as to promote adhesion. In further embodiments, fibrous chitosan is not treated with acetic acid, and instead is subjected to γ-irradiation in a nitrogen field, nitrogen-gas based plasma treatment, and/or another treatment method that increases the concentration of amino groups on and around the chitosan so as to increase wettability, hydrophilicity and mucoadhesion without exposure to the acetic acid after being formed into a fibrous fleece.

It is to be understood that further treatments may enhance chitosan-based textiles. For example, in one embodiment chitosan fibers are soaked in alcohol, preferably for about an hour. In experiments, such a treatment caused the chitosan fibers to be much whiter, but with no structure change of the chitosan fiber. The total bacterial count of the chitosan fibers was also reduced. Such treated textiles can then be further treated using γ-irradiation, plasma, or both.

In some embodiments, the endotoxin levels in the chitosan before electron beam sterilization (25 K Grey) are 29 EU/g. After electron beam sterilization the chitosan can have EU levels about 2 EU/g. However, the chitosan is oxidized which causes a reduction of mucoadhesion, molecular weight (MW), and solubility. Nitrogen plasma exposure results in surface nitrogenation of chitosan that may increase bioadhesive, hemostatic and anti-microbial activity.

In contrast to electron beam, non-thermal atmospheric nitrogen gas plasmas do not degrade thermo-labile chitosan and are the most efficient, least material-damaging reagents. Nitrogen plasma may be the ideal reagent for depyrogenating chitosan since it does not affect physical and functional properties and may, in fact, increase mucoadhesivity by addition of elemental nitrogen to chitosan surfaces.

Hydrogel with Chitosan

In some embodiments, the chitosan can be formed into a hydrogel material which can be used in similar applications as described with reference to the microfibril chitosan. A hydrogel is a gel in which the swelling agent is water or other liquid solution. Hydrogels can include a solid three-dimensional cross-linked network containing a dispersion of water molecules. Hydrogels can be inherently adhesive, and because of their significant water content, can possess a degree of flexibility very similar to natural tissues. Hydrogels can be formed of natural or synthetic polymers.

Chitosan can be used in hydrogels intended for internal surgical or medical uses. Chitosan's biocompatablity and mucoadhesive properties enable it to be used in a chitosan hydrogel material for an implantable device as described herein. In some embodiments, the chitosan material used in the hydrogel can be in the form of a chitosan powder, flake, fiber, and/or other chitosan materials known in the art. Chitosan materials for use in hydrogels possess the same purification restrictions as discussed herein with reference to chitosan fibers. The γ-irradiation in a nitrogen field, nitrogen-gas based plasma treatment, and/or other treatment methods to purify the chitosan can be utilized to ensure endotoxin removal suitable for the use of the chitosan for internal medical procedures or treatments. A depyrogenated chitosan hydrogel matrix can be produced. The chitosan hydrogel matrix can be implanted or injected into a target region. In some embodiments, the chitosan hydrogel matrix can include a medicament, therapeutic agent, or other agent.

### Chitosan Nanoparticles

Other forms of chitosan can be subjected to the chitosan purification treatment as described herein. Chitosan nanoparticles similar to those described in Applicants' copending International Patent Application No. PCT/US2013/30582, filed Mar. 12, 2013, and directed to a “SUBSTANCES AND METHODS FOR THE TREATMENT OF CEREBRAL AMYLOID ANGIOPATHY RELATED CONDITIONS OR DISEASES”, now published as WO 2013/138368. The entirety of this application is hereby incorporated by reference. Chitosan nanoparticles can have various applications as a drug delivery device and can be utilized to deliver various molecules to a targeted site. The biocompatablity of chitosan and the endotoxin removal techniques described herein allow chitosan nanoparticles to be effective targeted drug delivery devices that can be used in various applications.

The nanoparticles of various embodiments preferably have an average particle size of about 0.5, 0.6, 0.7, 0.8, 0.9, 1, 1.1, 1.2, 1.3, 1.4, 1.5, 1.6, 1.7, 1.8, 1.9, or 2 μm or more, e.g., 1 nm to 2000 nm or more. The preferred size may depend on the drug to be encapsulated or the condition to be treated. In other embodiments, average particle size may be less than about 0.5 μm (500 nm), or 400, 300, 200, 100, 90, 80, 70, 60, 50, 40, 30, 20, 10, 9, 8, 7, 6, 5, 4, 3, 2, 1 nm or less. In various embodiments, the particles are of a substantially uniform size distribution, that is, a majority of the particles present have a diameter generally within about ±50% or less of the average diameter, preferably within about ±45%, 40%, 35%, 30% or less of the average diameter, more preferably within ±25% or less of the average diameter, and most preferably within ±20% or less of the average diameter. The term “average” includes both the mean and the mode.

While a uniform size distribution may be generally preferred, individual particles having diameters above or below the preferred range may be present, and may even constitute the majority of the particles present, provided that a substantial amount of particles having diameters in the preferred range are present. In other embodiments, it may be desirable that the particles constitute a mixture of two or more particle size distributions, for example, a portion of the mixture may include a distribution on nanometer-sized particles and a portion of the mixture may include a distribution of micron-sized particles. The particles of various embodiments may have different forms. For example, a particle may constitute a single, integrated particle not adhered to or physically or chemically attached to another particle. Alternatively, a particle may constitute two or more agglomerated or clustered smaller particles that are held together by physical or chemical attractions or bonds to form a single larger particle. The particles can be in dry form, or in the form of a suspension in a liquid.

In some embodiments, chitosan in nanoparticulate form, e.g., solid form chitosan nanoparticles comprising therepeutic agents in solid form (e.g., as a tablet, capsule, or implant) or nanoparticles in liquid suspension or slurry (e.g., for oral administration, intravenous administration, or implantation by injection) can be provided. Chitosan nanoparticles can be made by spray-drying aqueous solutions or dispersions of chitosan and one or more pharmaceutically active components, optionally with a surface modifier to form a dry powder which consists of aggregated chitosan nanoparticles. An aqueous dispersion of chitosan, pharmaceutically-active agent and surface modifier, when spray dried, can form pharmaceutically-active agent embedded chitosan nanoparticles. In one embodiment, compositions are provided containing nanoparticles which have an effective average particle size of less than about 2000 nm, more preferably less than about 400 nm, less than about 300 nm, less than about 250 nm, less than about 100 nm, or less than about 50 nm, as measured by light-scattering methods. By “an effective average particle size of less than about 1000 nm” it is meant that at least 50% of the pharmaceutically-active agent particles have a weight average particle size of less than about 1000 nm when measured by light scattering techniques. Preferably, at least 70% of the pharmaceutically-active agent particles have an average particle size of less than about 1000 nm, more preferably at least 90% of the pharmaceutically-active agent particles have an average particle size of less than about 1000 nm, and even more preferably at least about 95% of the particles have a weight average particle size of less than about 1000 nm.

The compounds of various embodiments can be provided in the form of a spray-dried powder, either alone or combined with a freeze-dried nanoparticulate powder. Spray-dried or freeze-dried nanoparticulate powders can be mixed with liquid or solid excipients to provide unit dosage forms suitable for administration. Freeze dried powders of a desired particle size can be obtained by freeze drying aqueous dispersions of pharmaceutically-active agent and surface modifier, which additionally contain a dissolved diluent such as lactose or mannitol.

Milling of aqueous chitosan/pharmaceutically-active agent solutions to obtain chitosan nanoparticulates may be performed by dispersing pharmaceutically-active agent particles or dissolving a soluble pharmaceutically-active agent in a liquid dispersion medium comprising chitosan and applying mechanical means in the presence of grinding media to reduce the particle size of the pharmaceutically-active agent to the desired effective average particle size. The particles can be reduced in size in the presence of one or more surface modifiers. Alternatively, the particles can be contacted with one or more surface modifiers after attrition. Other compounds, such as a diluent, can be added to the pharmaceutically-active agent/surface modifier composition during the size reduction process. Dispersions can be manufactured continuously or in a batch mode.

Another method of forming a chitosan nanoparticle dispersion is by microprecipitation. This is a method of preparing stable dispersions of pharmaceutically-active agent and chitosan in the presence of one or more surface modifiers and one or more colloid stability enhancing surface active agents free of any trace toxic solvents or solubilized heavy metal impurities. Such a method comprises, for example, (1) dissolving chitosan and the pharmaceutically-active agent in a suitable solvent with mixing; (2) adding the formulation from step (1) with mixing to a solution comprising at least one surface modifier to form a clear solution; and (3) precipitating the formulation from step (2) with mixing using an appropriate nonsolvent. The method can be followed by removal of any formed salt, if present, by dialysis or diafiltration and concentration of the dispersion by conventional means.

In a non-aqueous, non-pressurized milling system, a non-aqueous liquid having a vapor pressure of about 1 atm or less at room temperature and in which the chitosan and pharmaceutically-active agent substance is essentially insoluble may be used as a wet milling medium to make a chitosan nanoparticulate/pharmaceutically-active agent composition. In such a process, a slurry of pharmaceutically-active agent and surface modifier may be milled in the non-aqueous medium to generate chitosan nanoparticulate/pharmaceutically-active agent particles. Examples of suitable non-aqueous media include ethanol, trichloromonofluoromethane, (CFC-11), and dichlorotetafluoroethane (CFC-114). An advantage of using CFC-11 is that it can be handled at only marginally cool room temperatures, whereas CFC-114 requires more controlled conditions to avoid evaporation. Upon completion of milling the liquid medium may be removed and recovered under vacuum or heating, resulting in a dry nanoparticulate composition.

In a non-aqueous, pressurized milling system, a non-aqueous liquid medium having a vapor pressure significantly greater than 1 atm at room temperature may be used in the milling process to make chitosan nanoparticulate/pharmaceutically-active agent compositions. The milling medium can be removed and recovered under vacuum or heating to yield a dry nanoparticulate composition.

Cryomilling is a variation of mechanical milling, in which powders or other solids are milled in a cryogen (usually liquid nitrogen, liquid carbon dioxide, or liquid argon) slurry or at a cryogenics temperature under processing parameters, so a nanostructured microstructure is attained. Cryomilling takes advantage of both the cryogenic temperatures and conventional mechanical milling. The extremely low milling temperature suppresses recovery and recrystallization and leads to finer grain structures and more rapid grain refinement. The embrittlement of the sample makes even elastic and soft samples grindable. Tolerances less than 5 μm can be achieved. The ground material can be analyzed by a laboratory analyzer. Freezer milling is a type of cryogenic milling that uses a solenoid to mill samples. The solenoid moves the grinding media back and forth inside a container, grinding the sample down to a desired degree of fineness. The idea behind using a solenoid is that the only moving part in the system is the grinding media inside the vial.

Spray drying is a process used to obtain a powder containing chitosan nanoparticulate/pharmaceutically-active agent particles following particle size reduction of the pharmaceutically-active agent in a liquid medium. In general, spray-drying may be used when the liquid medium has a vapor pressure of less than about 1 atm at room temperature. A spray-dryer is a device which allows for liquid evaporation and pharmaceutically-active agent powder collection. A liquid sample, either a solution or suspension, is fed into a spray nozzle. The nozzle generates droplets of the sample within a range of about 20 to about 100 μm in diameter which are then transported by a carrier gas into a drying chamber. The carrier gas temperature is typically between about 80 and about 200° C. The droplets are subjected to rapid liquid evaporation, leaving behind dry particles which are collected in a special reservoir beneath a cyclone apparatus.

If a liquid sample consists of an aqueous dispersion of chitosan nanoparticles and surface modifier, the collected product will consist of spherical aggregates of the chitosan nanoparticulate/pharmaceutically-active agent particles. If the liquid sample consists of an aqueous dispersion of nanoparticles in which an inert diluent material was dissolved (such as lactose or mannitol), the collected product will consist of diluent (e.g., lactose or mannitol) particles which contain embedded chitosan nanoparticulate/pharmaceutically-active agent particles. The final size of the collected product can be controlled and depends on the concentration of chitosan nanoparticulate/pharmaceutically-active agent and/or diluent in the liquid sample, as well as the droplet size produced by the spray-dryer nozzle.

In some instances it may be desirable to add an inert carrier to the spray-dried material to improve the metering properties of the final product. This may especially be the case when the spray dried powder is very small (less than about 5 μm) or when the intended dose is extremely small, whereby dose metering becomes difficult. In general, such carrier particles (also known as bulking agents) are too large to be delivered to the lung and simply impact the mouth and throat and are swallowed. Such carriers typically consist of sugars such as lactose, mannitol, or trehalose. Other inert materials, including non-chitosan polysaccharides and cellulosics, may also be useful as carriers.

Sublimation can be employed to obtain a chitosan nanoparticulate/pharmaceutically-active agent composition. Sublimation avoids the high process temperatures associated with spray-drying. In addition, sublimation, also known as freeze-drying or lyophilization, can increase the shelf stability of pharmaceutically-active agent compounds, particularly for biological products. Sublimation involves freezing the product and subjecting the sample to strong vacuum conditions. This allows for the formed ice to be transformed directly from a solid state to a vapor state. Such a process is highly efficient and, therefore, provides greater yields than spray-drying. The resultant freeze-dried product contains pharmaceutically-active agent and modifier(s).

### Therapeutic Agents

In some embodiments, the depyrogenated chitosan material can be prepared by any of the methods as described herein and can be implanted or injected into a target region with a medicament, therapeutic agent, or other agent. The medicament, therapeutic agent, or other agent can be incorporated into or mixed with the chitosan material. Depyrogenated chitosan can be used as an excipient for a variety of drugs. For example, therapeutic agents can include cytokine interleukin-12 (IL-12). IL-12 can have a significant anti-tumor and anti-metastatic effect. The IL-12 treatment is a cancer immunotherapy and can generate cancer immunity.

IL-12 augments natural killer (NK)/lymphocyte-activated killer cell activity, enhances cytolytic T cell generation, and induces interferon gamma (TNF-γ) secretion. IL-12 may provide significant protection against tumor re-challenge by potentiating immunologic memory and regulating T cell activity via proliferation of both activated CD4+ and CD8+T cell subsets.

Chitosan is a recognized drug delivery vehicle; however, it has not been utilized as a drug delivery vehicle clinically due to the high endotoxin levels of currently available materials. Additionally, IL-12 is a known immunotherapeutic agent; it has not been successfully applied clinically due to toxicity when given systemically. Chitosan is only used clinically as a topical hemostat because the material cannot be adequately depyrogenated for internal use with standard depyrogenation methods like ethylene oxide, γ-irradiation, heat, and/or electron beam without altering its advantageous functional properties. Thus, using the nitrogen plasma method as described herein to produce implantable, depyrogenated chitosan with unaltered functionality can enable chitosan and IL-12 to be used as a treatment for bladder cancer and chitosan to be used in many other ways clinically.

For example, in some embodiments, a depyrogenated chitosan hydrogel matrix can be produced as described above and the chitosan hydrogel matrix can be implanted or injected into a target region with a therapeutic agent. In some embodiments, the chitosan can be depyrogenated using γ-irradiation in a nitrogen field, nitrogen-gas based plasma treatment, and/or another treatment methods to purify the chitosan. The depyrogenated chitosan can be formed into a hydrogel. Therapeutic agents that can be incorporated or mixed with the chitosan hydrogel can include cytokine interleukin-12 (IL-12). IL-12 can be injected into, combined within, and/or seeded on the chitosan hydrogel. The chitosan hydrogel matrix can be implanted or injected into a target region with the therapeutic agent cytokine interleukin-12 (IL-12). The IL-12 can have significant anti-tumor and anti-metastatic effects in the target region.

Additionally, in some embodiments, a depyrogenated chitosan nanoparticle can be delivered to a target region with a medicament, therapeutic agent or other agent. For example, in some embodiments, a depyrogenated chitosan nanoparticle can be produced as described herein and in Applicants' copending International Patent Application No. PCT/US2013/30582, filed Mar. 12, 2013, and directed to a “SUBSTANCES AND METHODS FOR THE TREATMENT OF CEREBRAL AMYLOID ANGIOPATHY RELATED CONDITIONS OR DISEASES”, now published as WO 2013/138368. The entirety of this application is hereby incorporated by reference. The chitosan nanoparticle can be depyrogenated using γ-irradiation in a nitrogen field, nitrogen-gas based plasma treatment, and/or another treatment methods to purify the chitosan.

In some embodiments, the depyrogenated chitosan nanoparticle can include a targeting agent that allows for targeted delivery of the chitosan nanoparticle to the treatment site. Additionally, in some embodiments, a depyrogenated chitosan nanoparticle can be implanted or injected into a target region. The depyrogenated chitosan nanoparticle can be delivered to the target region with a medicament, therapeutic agent, or other agent. For example, depyrogenated chitosan nanoparticle can be delivered to the target region with a therapeutic agent. The therapeutic agents can be incorporated into or encapsulated within the chitosan nanoparticle. The therapeutic agent can include cytokine interleukin-12 (IL-12). The chitosan nanoparticle can be implanted or injected into a target region with the therapeutic agent cytokine interleukin-12 (IL-12). The IL-12 can have significant anti-tumor and anti-metastatic effects in the target region.

### Intravesical Delivery Vehicles

In some embodiments, depyrogenated chitosan devices can be purified and thereby used in internal medical applications including intravesical delivery vehicles. Development of an immunotherapeutic treatment for superficial bladder cancer using interleukin-12 (IL-12) with chitosan (Ch+IL-12) as a delivery vehicle can be achieved by reducing the IL-12 toxicity, chitosan endotoxin contamination, and achieving controlled paracrine IL-12 tumor delivery. The ability to make an implantable depyrogenated chitosan can allow this immunotherapeutic approach for treating superficial bladder cancer to be utilized in humans.

The outlook for patients with urinary bladder cancer is poor and new therapeutic approaches are needed. Urinary bladder cancer, the fifth most common cancer in the United States with over 70,000 new cases each year, is the most expensive cancer to treat per patient. Over 90% of bladder cancers are transitional cell carcinomas that present as superficial bladder tumors (stages Ta, Tis, or T1). The current standard-of-care for superficial bladder cancer is transurethral resection of the bladder tumor with post-operative “immunotherapy” provided by inducing an intravesicular infection with Bacillus Calmette-Guerin (BCG). Of patients treated with BCG, 20-30% of cases do not respond to BCG treatment and 30-50% develop recurrent tumors within five years. Despite the poor long-term protection against bladder tumor recurrence with BCG, no chemotherapeutic agent, including mitomycin C, has been shown to be superior. The immunotherapeutic approach as described herein can be based on providing a depyrogenated chitosan IL-12 formulation that has the simplicity and versatility necessary for immediate clinical testing.

Though the cytokine interleukin-12 (IL-12) has potent anti-tumor and anti-metastatic effects, there is significant dose and schedule dependent human toxicity. The systemic bolus administration of IL-12 to humans in cancer treatment trials resulted in severe toxicity and deaths. These adverse effects have resulted in an active search for alternative IL-12 delivery vectors that reduce toxicity and enhance adjuvant activity.

Specifically, results obtained in a pre-clinical murine bladder cancer model at the National Cancer Institute (NCI) demonstrated exceptional cure rates (88-100%) and long-term immunity against cancer re-challenge after 3 intravesical infusions of Ch+IL-12 but not IL-12 alone, chitosan alone, or the standard of care, Bacillus Calmette-Guérin (BCG), alone. Similarly encouraging results have been shown for other cancers including breast, colon, and pancreatic, meaning this combination may be useful for multiple cancers. The NCI study reported the remarkable therapeutic effectiveness of intravesical chitosan and IL-12 for curing the MB-49 mouse bladder cancer model. 100% of tumor bearing mice had complete tumor eradication after three intravesical treatments with chitosan and IL-12, but not with IL-12 alone or the standard of care (BCG) and, importantly, the “cured” mice rejected a cancer cell re-challenge. In other words, the mice developed immunity to superficial bladder cancer after 3 intravesical treatments with chitosan and IL-12.

Because of significant pyrogen contamination in the chitosan element the implantation into humans has been prohibited. The devices and methods described herein include a process for depyrogenating chitosan with non-thermal atmospheric nitrogen gas plasma (NtANP) that reduces chitosan endotoxins and may enhance functional properties such as mucoadhesion and controlled paracrine drug delivery. Such alterations to the chitosan material can make the material suitable for internal use in humans.

Chitosan can be a promising delivery vehicle for IL-12 and can be used in studies similar to those performed at the National Cancer Institute (NCI) showing 88-100% cure rates and long-term immunity against cancer recurrence in a mouse model of bladder cancer. In some embodiments, chitosan can be a good delivery vehicle because delivery of IL-12 with cationic chitosan can enhance anti-tumor effects while reducing systemic toxicity of IL-12. For example, chitosan can reduce the systemic toxicity through its mucoadhesion properties by keeping the cytokine localized to the target site via adhesion to the anionic bladder wall by electrostatic forces. In some embodiments, chitosan can reduce the systemic toxicity by enhancing transmucosal passage by loosening gap junctions in the bladder wall.

Intravesical drug delivery for bladder cancer faces the challenge of maintaining drug residence levels despite urine collection and voiding. Chitosan can overcome this challenge through strong adhesion to the bladder wall. The mucoadhesion properties of depyrogenated chitosan can allow the chitosan to remain in the target area of the bladder wall. The endotoxin limits for implanted medical devices are ≦0.5 EU (endotoxin unit)/g or ≦20 EU/device. The limits should be analyzed based on the treatment area and dilution of the device. For example, in an embodiment for the intravesical drug delivery for bladder cancer, since the maximum volume a human bladder typically holds is 600 mL and chitosan is mixed with IL-12 as a 1% hydrogel, a maximum of 6 g of chitosan will be used in any one dose of chitosan and IL-12. This means that the chitosan in that embodiment can contain 3.33 EU/g ((20 EU/device)÷(6 g/device)=3.33 EU/g). These levels are far exceeded by currently available “medical grade” chitosan. For example, EU levels in “medical grade” chitosan measured by independent laboratories are as follows: Sanli chitosan (Chinese)=620 EU/g; Nova Matrix, Protasan G-213 (Norway) reported by the company as ≦100 EU/g, analyzed by another laboratory 247 EU/g, Scion Cardio-Vascular, Inc. (U.S.A.) chitosan=29 EU/g (1 g=1 device). Scion Cardio-Vascular's chitosan has the lowest endotoxin concentration, but still requires further depyrogenation for FDA approval to implant.

Though chitosan is considered a safe pharmaceutical excipient, there are now conflicting reports regarding the polymer's biocompatibility. The use of a zwitterionic chitosan derivative (ZWC) as an alternative to chitosan for certain medical devices has been proposed since the ZWC has greater solubility at physiologic pH and does not elicit an immune response despite containing orders of magnitude more endotoxins than chitosan as shown in the table below.

| Sample | Endotoxin concentration EU/gm) |
|---|---|
| Chitosan glutamate | 247 |
| Glycol chitosan | 311 |
| LMCS (precursor low M.W. chitosan) | 311 |
| ZWC (An/Am = 0.3) | 6,860 |
| ZWC (An/Am = 0.7) | 14,150 |

LMCS = precursor low M.W. chitosan;
ZWC = Zwitterionic chitosan derivative;
An/Am = Anhydride/amine ratio

ZWC is not an acceptable alternative to chitosan for use as a delivery vehicle for IL-12 to the bladder wall, despite its ability to sequester endotoxins, for several reasons. First, chitosan as a delivery vehicle for IL-12 to the bladder wall requires chitosan to be cationic so that it will adhere to the anionic bladder wall via electrostatic forces. The pKa characteristics of ZWC make achieving a cationic charge difficult. Secondly, the ZWC will eventually be metabolized to sugars and oligosaccharides thereby releasing the ZWC-bound endotoxins, which are extraordinarily high in number. This release of endotoxins will cause a massive inflammatory reaction. Past studies including ZWC did not observe an inflammatory reaction to the ZWC because they only followed the animals for 7 days after implanting the ZWC, which is not long enough for chitosan to be significantly metabolized.

Thirdly, ZWC is made from low molecular weight chitosan, meaning the ZWC will also possess a low molecular weight. This will cause problems with the viscosity of the chitosan IL-12 hydrogel. Fourthly, a brief cost analysis indicates the plasma treatment of already available chitosan is more cost effective than the synthesis of GMP-grade ZWC. Fifthly, chitosan is a well-known, well-characterized material whereas ZWC is not. For example, there is no published data describing the effect of ZWC on gap junctions in the bladder wall. Finally, it is unknown how ZWC interacts with IL-12, a key variable in determining the effectiveness of the Ch+IL-12 treatment. The chitosan used in the NCI studies was chitosan glutamate (Protasan UP G113, molecular weight (M.W.) to 200 kDa, degree of deacetylation 75.90%, obtained from Novamatrix (Norway)). Endotoxin content according to the manufacturer is ≦100 EU/g, but was reported by other sources to be 247 EU/g. The high endotoxin levels did not allow the studies to proceed for internal implantation in humans.

In some embodiments, depyrogenated chitosan may not only solve the endotoxin contamination of chitosan but may also enhance mucoadhesive properties to the bladder wall for controlled delivery of IL-12 to the tumor. Additionally, in some embodiments, successful depyrogenation of chitosan can eliminate the inflammatory and immunologic artifacts secondary to endotoxins.

The intravesical drug delivery of IL-12 by chitosan hydrogel for bladder cancer therapy has immediate translational significance since this depyrogenated chitosan-based intravesicular delivery of IL-12 is versatile, simple, and readily applied in a clinical setting. The clinical utilization of chitosan, previously hindered by pyrogen contamination, can meet other biomedical needs, for example, tissue scaffolding, drug and gene delivery, or an implantable hemostat. The depyrogenation of the chitosan material by nitrogen plasma will lower endotoxin levels in chitosan hydrogel to ≦0.5 EU/g or ≦20 EU/device, a level that will enable FDA approval for human bladder implantation as well as enabling other internal and/or implantable chitosan biomaterial for biomedical applications as described herein or known in the art.

### Example I

### Chitosan Depyrogenation and Endotoxin Reduction

Nitrogen plasma dosimetry to achieve chitosan depyrogenation [(≦0.5 EU/g or ≦20 EU/device)] is based on a “substerilization incremental dose” protocols. Power and time variables of the plasma enable survivor curves for bacterial bioburdens (Colony Forming Units (CFUs)/g and endotoxins EU/g).

CFUs and EUs plotted on the log vertical scale and plasma exposure (time power) on the linear horizontal scale enable calculation of the log [N(t)/No]=k·t relationship (No is initial concentration of CFUs/EUs, Nt the concentrations found at given time and power, k is the endotoxin “death rate” constant), and t=Time to reduce the original CFU/EU concentration by 90% as a one log 10. A 6 log 10 reduction is necessary to secure FDA approval for implantation based on ISO 10993 series standards. An example of chitosan endotoxin reduction before and after nitrogen plasma treatment conducted with procedures described herein is given in the table below.

| Material | EU/g |
|---|---|
| Chitosan I Chinese before plasma | 620.5 |
| Chitosan Chinese γ-irrad. + N2 plasma (5 min) | 52.8 |
| **Chitosan Chinese Fabric γ-irrad. + N2 plasma (10 min) | 12.7 |
| ***Shrimp Lotox Chitosan (powder) | <65 |

*Endotoxin levels determined by Steri-Pro Labs, Ontario, CA
**FDA endotoxin requirements for an implantable medical device (≦20 EU/device)
***Shrimp Lotox ™, “ultrapure chitosan,” from Syndegen, Claremont, CA (material not available for commercial sale), M.W. 160-312 kDa

The endotoxin reduction goal for the chitosan is ≦0.5 EU/g (the equivalent of 20 EU/device assuming a 600 mL 1% chitosan max dose), a level that will enable the FDA to approve the material for human implantation. In some embodiments, endotoxin levels may not be reduced to this level by increasing nitrogen plasma dosing. Therefore, in some embodiments, the nitrogen plasma treatment is augmented with gamma irradiation.

Though not expected, the depyrogenation process may introduce changes in the physical and chemical properties of the chitosan molecule. Therefore the molecular weight and mucosal adhesion as well as other properties are tested. However, should the chitosan be altered in its physical and chemical properties inhibiting functions, the chitosan nitrogen plasma treatment can be adjusted to depyrogenate chitosan to avoid altering important functional properties.

### Example II

### Testing Depyrogenated Chitosan for Effect on Mucoadhesion Properties in the Course of Nitrogen Plasma Depyrogenation

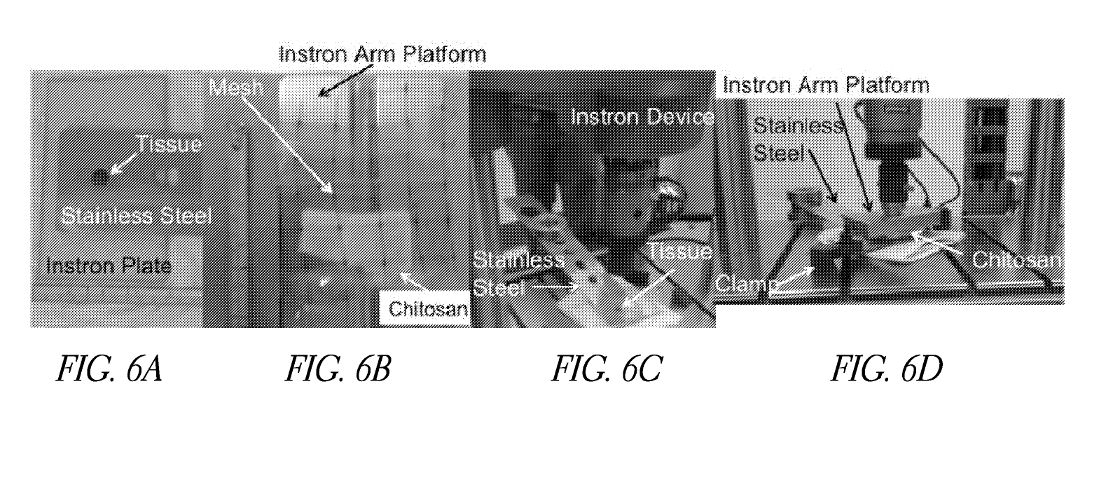

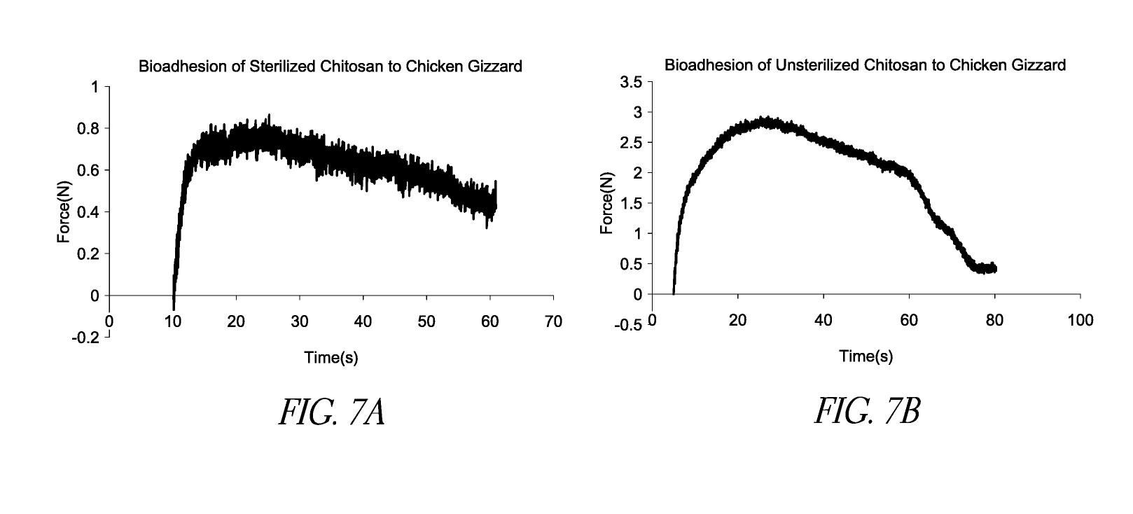

To test that the depyrogenation process does not introduce any significant changes in the physical and chemical properties of chitosan, molecular weight and mucosal adhesion is tested. Since mucoadhesion plays a critical role in paracrine Ch+IL-12 delivery, a test procedure is utilized to determine this property after depyrogenation procedures have been performed on chitosan. For example, apparatus that can be employed to measure chitosan mucoadhesivity is shown in FIGS. 6A-D. In a preliminary study, chitosan was tested for adhesion to the surface of a chicken gizzard before and after e-beam sterilization at 25 KGrey. As shown in FIGS. 7A-B, sterilization caused a loss of molecular weight from 600 KDa to 200 KDa and a corresponding loss of mucoadhesivity by −67%. The chitosan appeared yellowish-brown after 25 KGrey exposures, the solubility was impaired, and endotoxin levels were reduced from 29 EU/g to 1.3-1.8 EU/g.

FIGS. 6A-D illustrate an Instron setup for testing of bioadhesivity of chitosan before and after electron beam sterilization. This set up is shown in FIGS. 6A and 6C. Chicken gizzard obtained from a local supermarket was placed under a 1 mm thick piece of stainless steel with a circular cutout approximately 3.5 cm in diameter to expose a standard surface area of tissue. The stainless steel and tissue were together secured to the lower plate of the Instron device via (FIG. 6A) tape and (FIG. 6D) clamps. FIG. 6B illustrates how a sterilized or unsterilized chitosan device was secured to the platform of the moveable arm of an Instron tensiometer via a thin mesh that was taped to the arm itself.

FIGS. 7A-B show the bioadhesion of chitosan to chicken gizzard measured in force (N). The force required to remove an electron beam sterilized chitosan device is shown in FIG. 7A. The force required to remove an unsterilized chitosan device is shown in FIG. 7B. The force required to remove electron beam sterilized chitosan and unsterilized chitosan was measured using an Instron tensiometer with the setup depicted and described in FIGS. 6A-D. Chitosan was brought into contact with the tissue and either 30 N (FIG. 7B, data not shown) or 60 N (FIG. 7A, data not shown) of force was applied and held for 30 seconds. The chitosan was then pulled away from the tissue at a rate of 2 mm/s and the force required to do so was monitored and plotted graphically. The sterilized chitosan required <1.0 N of force to remove the chitosan from the tissue whereas the unsterilized chitosan required approximately 3.0 N of force to remove the chitosan from the tissue. This demonstrated that electron beam sterilization reduced the bioadhesivity of chitosan by approximately 67%.

The method developed to measure chitosan mucoadhesivity as described with reference to FIGS. 6A-D and 7A-B is also used to test for changes in the physical and chemical properties of chitosan mucosal adhesion introduced as a result of the depyrogenation process. The mucoadhesive studies are repeated on fresh pig bladders with varying nitrogen plasma and/or gamma-irradiated chitosan hydrogels to establish clinical parameters.

### Example III

### Testing Depyrogenated Chitosan for Effect on Endotoxins in the Course of Nitrogen Plasma Depyrogenation

The endotoxin content of the chitosan is determined by the Endosafe® Portable Test System (PTS™), a chromogenic Limulus Amebocyte Lysate (LAL) assay. The Endosafe® system gives reliable and precise results within 15 minutes, a marked improvement over the time and complexities of traditional LAL assays that are demanding and subject to inhibition/enhancement artifacts.

The FDA has approved the Charles River Laboratories Portable Test System (PTS) for endotoxin assays. Chitosan samples (before and after controlled nitrogen plasma depyrogenation) are tested as viscous 1% (w/o) hydrogel in LAL reagent water [adjusted to pH 6.5]. The PTS system can detect endotoxin levels from 0.005 to 10 EU/g with built-in “spike” recovery for every test, and has been validated in other laboratories and has the advantages of speed and simplicity.

### Example IV

### Testing Depyrogenated Chitosan for Effect on Molecular Weight in the Course of Nitrogen Plasma Depyrogenation

The effect of nitrogen plasma on the chitosan material is determined by post-treatment molecular weight determinations. Size exclusion or gel permeation chromatography is an accurate and reproducible method for determining the molecular weight of chitosan, including its polydispersity. Polydispersity describes the breadth of the chitosan molecular weight distribution and cannot be easily obtained from intrinsic viscosity data. The nature of the polydispersity has a profound effect upon the solution properties and hence the processability of chitosan into shaped objects such as fibers and films. A Waters 150-C ALC/GPC chromatograph (Waters Chromatography Div., Millipore Corp., Milford, Mass.) will be used. Pullan standards (Showa Denko Co., Tokyo) of the appropriate MW are used to calibrate the column.

### Example V

### Testing Depyrogenated Chitosan for Elemental Nitrogen in the Course of Nitrogen Plasma Depyrogenation

The micro Kjeldahl nitrogen determination on the chitosan before and after nitrogen plasma treatment is correlated with endotoxin levels, mucoadhesivity and plasma dosimetry.

### Example VI

### Testing Depyrogenated Chitosan and IL-12: Orthotopic Bladder Cancer Models

A murine bladder cancer model can be conducted to ensure no loss of efficacy in the chitosan and IL-12 treatment when non-thermal atmospheric nitrogen gas plasma (NtANP) depyrogenated chitosan is used.

Female mice (10-12 weeks old) have orthotopic bladder cancers induced by the intravesicular placement of MB-49 cells. MB-49 cells are provided by the NCI by a Material Transfer Agreement. The cancer shares similar properties with human bladder cancers (cell surface markers, immunologic profile). For a tumor cell to “take” it is necessary to prep the bladder mucosa with either ethanol or poly-L-lysine (PLL). 100 μl of 0.1 mcg/ml of PLL M.W. 70,000-150,000 is introduced into the bladder and held in place by clamping the catheter for 10 minutes to enhance tumor cell adherence. A 100 μl solution containing 2×106 MB-49 cells is introduced into the PLL prepped bladder and held in place for 45 minutes. Since there is a dead space of 50 μl in the catheter only 1×106 MB-9 cells and 50 μl of PLL reach the bladder mucosa. Mice are anesthetized for all procedures: catheterization, bladder preparation, instillation of MB-49 cells, intravesicular treatment, and blood draws. Orthotopic bladder cancers develop in 100% of implanted animals within one week and are fatal within 40 days.

100 tumor-bearing animals are divided into 5 animal study groups of 20 animals each. 5 different treatments are initiated as intravesicular instillations on days 7, 14, 21 and 28 post-tumor implantation. Mice in the cured group (group D) will be re-challenged with a tumor cell implant at 60 days.

Group A—treatment with 100 μl PBS
Group B—100 μl NtANP depyrogenated chitosan, 1% solution (1:10 w/v)

### Group C—IL-12, 5 mcg in 100 μl PBS

Group D—1% NtANP depyrogenated Ch+IL-12, 5 mcg in 100 μl PBS
Group E—1% NtANP depyrogenated chitosan+1.35 mg of BCG in 100 μl PBS.
Group F—“Cured” mice, survival from group D in a tumor rechallenge at 60 days
Group G—20 control mice—no tumors implants. 10 mice 1.35 mg BCG in 1% chitosan, 10 mice PBS in 1% chitosan solution. The control mice will be allowed to survive 60 days, then sacrificed for histologic study to compare the bladder inflammatory responses.

Mice with MB-49 mouse bladder cancers do not survive more than 40 days after tumor cell instillation without treatment (Groups A, B, E). 100% survival is anticipated at 60 days with the chitosan/IL-12 treatment (Group D) and 60% survival at 60 days with IL-12 alone (Group C). Survival is based on both time to euthanasia as determined by humane criteria and death. Signs for imminent death include hematuria, weight loss, a hunching habitus, and other distress signals—dull fur, apathy, and visible signs of a growing tumor. Distressed animals are euthanized since they may die suddenly of uremia secondary to occlusion of urethral and ureteral orifices and be cannibalized. Animals are euthanized in a CO2 chamber and an immediate post-mortem is performed with the bladder removed as well as a search for metastases within the lungs or abdomen. The bladder is fixed in both 10% buffered neutral formalin and embedded in OCT for immunohistochemical identification of inflammatory cells (CD3, CD4, CD8A and F4/80) to further define the cellular response to tumor and treatment.

Mice surviving long-term (>60 days), following treatments of NtANP chitosan plus IL-12 (Group D) or IL-12 (Group C) are intravesically rechallenged with the MB-49 tumor cells to test immunologic memory. Cancer naïve mice are challenged under the same conditions. Serum cytokines are assayed including: IL-12, p70, IFN-γ, TNF-α, and IL-6 24 h with serum alanine aminotransferase activity measured as a sign of IL-12 toxicity.

The study includes 7 groups of 20 animals each. The primary outcome is survival to 60 days. The hypothesis is that the 60-day mortality for groups A, B and E will be 100%, for group C will be 40% and groups D and G will be 0%. For these extreme expected differences in mortality, 20 animals in each group provide greater than 95% power (alpha=0.05) to detect a statistically significant difference in mortality between the groups with and without treatment. A z-test for proportions with a correction for multiple testing is used to compare mortality between groups. An additional outcome is histology +/− for cancer. It is expected that the cancer outcome closely follows the mortality outcome.

While various embodiments of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. Thus the breadth and scope of the present disclosure should not be limited by any of the above-described exemplary embodiments.

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

The following numbered items provide further disclosure forming part of the present application.

1. A method of making a drug delivery device, comprising:
irradiating endotoxin-containing chitosan under a nitrogen plasma in a presence of γ-irradiation, whereby an amount of endotoxins present in the chitosan is reduced;
forming the irradiated chitosan into a hydrogel material; and
combining a therapeutic agent with the hydrogel material, whereby a drug delivery device is obtained, wherein an amount of endotoxins present in the drug delivery device is less than 20 E.U. per device or less than 0.5 E.U. per gram.

2. The method of 1, wherein irradiating is conducted at ambient temperature.

3. The method of any of 1-2, wherein after the irradiating the chitosan is not substantially reduced in molecular weight.

4. The method of any of 1-3, wherein the irradiating is conducted under γ-irradiation at 25 kGy for 15 hours.

5. The method of any of 1-4, wherein the nitrogen-based plasma consists essentially of nitrogen plasma.

6. The method of any of 1-4, wherein the nitrogen-based plasma consists of nitrogen plasma.

7. The method of any of 1-6, wherein the therapeutic agent is interleukin-12.

8. The method of any of 1-7, wherein the therapeutic agent is injected into the hydrogel material.

9. The method of any of 1-7, wherein the therapeutic agent is combined with the hydrogel material prior to swelling of the gel.

10. A method of making a drug delivery device, comprising:
irradiating endotoxin-containing chitosan under a nitrogen plasma in a presence of γ-irradiation, whereby an amount of endotoxins present in the chitosan is reduced;
forming the irradiated chitosan into a nanoparticle; and
encapsulating a therapeutic agent within the nanoparticle, whereby a drug delivery device is obtained, wherein an amount of endotoxins present in the device is less than 20 E.U. per device or less than 0.5 E.U. per gram.

11. The method of 10, wherein the therapeutic agent is interleukin-12.

12. The method of any of 10-11, wherein irradiating is conducted at ambient temperature.

13. The method of any of 10-12, wherein after the irradiating the chitosan is not substantially reduced in molecular weight.

14. The method of any of 10-13, wherein the irradiating is conducted under γ-irradiation at 25 kGy for 15 hours.

15. The method of any of 10-14, wherein the nitrogen-based plasma consists essentially of nitrogen plasma.

16. The method of any of 10-14, wherein the nitrogen-based plasma consists of nitrogen plasma.

17. A pharmaceutical composition, comprising:
a hydrogel of chitosan; and
a therapeutic agent, wherein the hydrogel is configured to deliver the therapeutic agent to a target tissue, and wherein an amount of endotoxins present in the pharmaceutical composition is less than 0.5 E.U. per gram.

18. The pharmaceutical composition of 17, wherein the therapeutic agent is interleukin-12.

19. The pharmaceutical composition of any of 17-18, wherein the chitosan of the hydrogel is derived from an endotoxin-containing chitosan that is irradiated under a nitrogen plasma in a presence of γ-irradiation so as to reduce the amount of endotoxins present in the device to less than 20 E.U. per device or 0.5 E.U. per gram.

20. The pharmaceutical composition of any of 17-19, wherein the nitrogen plasma consists essentially of nitrogen plasma.

21. The pharmaceutical composition of any of 17-20, wherein a molecular weight of the chitosan is not substantially reduced upon irradiation.

22. The pharmaceutical composition of any of 17-21, wherein an average molecular weight of the chitosan is not reduced more than 5% upon irradiation.

23. A drug delivery device comprising the pharmaceutical composition of any of 17-22, wherein an amount of endotoxins present in the drug delivery device is less than 20 E.U. per device.

1. A method of making a drug delivery device, comprising:
irradiating endotoxin-containing chitosan under a nitrogen based plasma in a presence of γ-irradiation, whereby an amount of endotoxins present in the chitosan is reduced;
forming the irradiated chitosan into a hydrogel material; and
combining a therapeutic agent with the hydrogel material, whereby a drug delivery device is obtained, wherein an amount of endotoxins present in the drug delivery device is less than 20 E.U. per device or less than 0.5 E.U. per gram.

2. The method of claim 1, wherein irradiating is conducted at ambient temperature.

3. The method of claim 1, wherein after the irradiating the chitosan is not substantially reduced in molecular weight.

4. The method of claim 1, wherein the irradiating is conducted under γ-irradiation at 25 kGy for 15 hours.

5. The method of claim 1, wherein the nitrogen-based plasma consists essentially of nitrogen plasma.

6. The method of claim 1, wherein the nitrogen-based plasma consists of nitrogen plasma.

7. The method of claim 1, wherein the therapeutic agent is interleukin-12.

8. The method of claim 1, wherein the therapeutic agent is injected into the hydrogel material.

9. The method of claim 1, wherein the therapeutic agent is combined with the hydrogel material prior to swelling of the gel.

10. A method of making a drug delivery device, comprising:
irradiating endotoxin-containing chitosan under a nitrogen based plasma in a presence of γ-irradiation, whereby an amount of endotoxins present in the chitosan is reduced;
forming the irradiated chitosan into a nanoparticle; and
encapsulating a therapeutic agent within the nanoparticle, whereby a drug delivery device is obtained, wherein an amount of endotoxins present in the device is less than 20 E.U. per device or less than 0.5 E.U. per gram.

11. The method of claim 10, wherein the therapeutic agent is interleukin-12.

12. The method of claim 10, wherein irradiating is conducted at ambient temperature.

13. The method of claim 10, wherein after the irradiating the chitosan is not substantially reduced in molecular weight.

14. The method of claim 10, wherein the irradiating is conducted under γ-irradiation at 25 kGy for 15 hours.

15. The method of any of claim 10, wherein the nitrogen-based plasma consists essentially of nitrogen plasma.

16. The method of claim 10, wherein the nitrogen-based plasma consists of nitrogen plasma.

17. A pharmaceutical composition, comprising:
a hydrogel of chitosan; and
a therapeutic agent, wherein the hydrogel is configured to deliver the therapeutic agent to a target tissue, and wherein an amount of endotoxins present in the pharmaceutical composition is less than 0.5 E.U. per gram.

18. The pharmaceutical composition of claim 17, wherein the therapeutic agent is interleukin-12.

19. The pharmaceutical composition of claim 17, wherein the chitosan of the hydrogel is derived from an endotoxin-containing chitosan that is irradiated under a nitrogen based plasma in a presence of γ-irradiation so as to reduce the amount of endotoxins present in the device to less than 20 E.U. per device or 0.5 E.U. per gram.

20. The pharmaceutical composition of claim 19, wherein the nitrogen based plasma consists essentially of nitrogen plasma.

21. The pharmaceutical composition of claim 17, wherein a molecular weight of the chitosan is not substantially reduced upon irradiation.

22. The pharmaceutical composition of claim 17, wherein an average molecular weight of the chitosan is not reduced more than 5% upon irradiation.

23. A drug delivery device comprising the pharmaceutical composition of claim 17, wherein an amount of endotoxins present in the drug delivery device is less than 20 E.U. per device.
